(19) United States
(12) Patent Application Publication
(10) Pub. No.: US 2021/0161866 A1
(43) Pub. Date: Jun. 3, 2021

(54) DRUG FOR USE IN COMBINATION, AND USE THEREOF FOR PREPARING DRUGS FOR TREATMENT OF HIGH-GRADE BRAIN TUMORS IN RESPONSE TO INEFFECTIVE STANDARD TREATMENT FOR POSTOPERATIVE RECURRENCE

(71) Applicant: SICHUAN JIUZHANG BIOLOGICAL SCIENCE AND TECHNOLOGY CO., LTD., Chengdu, Sichuan (CN)

(72) Inventors: Jie ZHANG, Chengdu, Sichuan (CN); Xiaoguang CHEN, Chengdu, Sichuan (CN); Wenbin LI, Chengdu, Sichuan (CN); Huarong YANG, Chengdu, Sichuan (CN)

(21) Appl. No.: 17/053,037

(22) PCT Filed: Apr. 30, 2019
(86) PCT No.: PCT/CN2019/085092

§ 371 Date: Feb. 16, 2021

(30) Foreign Application Priority Data
May 4, 2018 (CN) 201810421557.0

(51) Int. Cl. A61K 31/4188; A61K 31/216; A61K 9/00; A61P 35/00

(52) U.S. Cl. CPC A61K 31/4188; A61K 31/216; A61P 35/00; A61K 9/0053; A61K 9/0019

(57) ABSTRACT

A drug for use in combination for treating high-grade brain tumors in response to ineffective standard treatment for postoperative recurrence. The drug for includes chlorogenic acid and temozolomide of the same or different unit preparations for simultaneous or separate administration, and a pharmaceutically acceptable carrier. A pharmaceutical composition includes chlorogenic acid and temozolomide can be used to prepare a drug for treatment of high-grade brain tumors for which standard treatment for postoperative recurrence is ineffective.

### TECHNICAL FIELD

The present invention particularly relates to a drug combination and its use in preparation of a drug for treatment of high-grade brain tumors that recur after surgery and are ineffective for standard therapy.

### BACKGROUND ART

Brain tumors, also known as intracranial tumors and craniocerebral tumors, denote neurological tumors that occur in the cranial cavity, including tumors originating from neuroepithelium, peripheral nerves, meninges, and germ cells, tumors of lymphoid and hematopoietic tissues, as well as craniopharyngiomas and granulosa cell tumors in sella turcica area. According to the “World Health Organization Central Nervous System (CNS) Tumor Classification (2016)”, which integrates tissue phenotype and gene phenotype CNS tumor classification principles, brain tumors include diffuse astrocyte and oligodendrocyte tumors, and other 17 types of tumors, including astrocytic tumors, ependymal tumors, choroid plexus tumors, neurons and mixed neuronal-glial tumors, pineal gland tumors, embryonic tumors and so on. High-grade brain tumors denote brain tumors of grades III and IV in the WHO classification of central nervous system tumors, such as glioblastoma, diffuse midline glioma, pineal blastoma, medulloblastoma, and malignant nerve sheath tumor, anaplastic astrocytoma, anaplastic oligodendroglioma, etc.

The high fatality rate of brain tumors is a major factor threatening human life. Currently, the general methods of treating brain tumors include surgery, chemotherapy, and radiotherapy. For high-grade brain tumors, most of them require surgery. After surgery, some patients' conditions improve and will not develop further. Some patients will relapse after surgery. Although the recurrent patients can be treated with standard therapy (including radiotherapy and chemotherapy), the conditions of almost all patients cannot be effectively controlled, and the lesions will progress, resulting in the inefficiency of standard therapy.

Said high-grade brain tumors that recur after surgery and do not respond to standard therapy account for about 50% of adult primary malignant brain tumors. The main pathological types include glioblastoma, anaplastic astrocytoma, anaplastic oligodendrocytoma, anaplastic oligodendroastrocytoma, etc. Among them, the incidence of malignant glioma has been increasing in recent years, especially glioblastoma has the highest incidence, accounting for about 46.1% of primary malignant central nervous system tumors, and increases as age. The incidence is highest in the elderly. Malignant gliomas are mostly located in the cerebral hemispheres, grow quickly, and the space-occupying effect is obvious. It appears as a mixed density mass on MRI and presents uneven ring enhancement, accompanied by an edema zone. Its main clinical manifestations are symptoms of intracranial hypertension, namely headache, nausea and vomiting, visual disturbances, etc., accounting for about 90% of all cases. Others include mental changes, seizures, focal neurological symptoms and the same. The severity of symptoms is related to the tumor size, the location, and the age of the patient and so on.

Malignant high-grade brain tumors that recur after surgery and do not response to standard therapy have the characteristics of rapid progress, high mortality, etc. Taking glioblastoma as an example, the standard treatment scheme is to maximize surgical resection under the premise of ensuring neurological function. After surgery, local radiotherapy was given, accompanied by chemotherapy using temozolomide (100 mg/m²) via oral administration, followed by 6 cycles of adjuvant chemotherapy using temozolomide (150-200 mg/m²). As the use of standard treatment plan, the median survival period can reach 14 months. However, due to its high degree of malignancy, strong invasiveness, invasive growth and other biological characteristics, it is difficult to completely resect the tumors by surgery, and residual tumor cells make malignant high-grade brain tumors that recur after surgery and do not respond to standard treatments are very likely to relapse. The prognosis of malignant glioma after recurrence is poor, with a median survival time of only about 7 months. There is no effective chemotherapy regimen for high-grade brain tumors that recur after surgery and do not respond to standard treatments.

### CONTENT OF THE INVENTION

In order to solve above problems, the present invention provides a drug combination and a pharmaceutical composition.

The present invention provides a drug combination for treatment of high-grade brain tumors that recur after surgery and do not response to standard treatment, that contains unit preparations of the same or different specifications, used for simultaneous or separate administration of chlorogenic acid and temozolomide, as well as pharmaceutically acceptable carrier.

Wherein, the mass ratio of chlorogenic acid and temozolomide is 35:100-350:100.

Further, the mass ratio of chlorogenic acid and temozolomide is 85:100-200:100; the mass ratio of chlorogenic acid and temozolomide is 85:100-175:100; preferably, the mass ratio of chlorogenic acid and temozolomide is 100:100 or 200:100.

Wherein, the preparation is an oral preparation or an injection preparation.

The present invention further provides the use of the drug combination mentioned above in the preparation of drugs for treatment of high-grade brain tumors that recur after surgery and do not response to standard treatment.

Wherein, the brain tumors include glioblastoma, anaplastic astrocytoma, anaplastic oligodendrocytoma, and anaplastic oligodendroblastoma.

The present invention further provides a pharmaceutical composition, that contain chlorogenic acid and temozolomide.

Wherein, the mass ratio of chlorogenic acid and temozolomide is 35:100-350:100.

Wherein, the mass ratio of chlorogenic acid and temozolomide is 85:100-200:100; the mass ratio of chlorogenic acid and temozolomide is 85:100-175:100; preferably, the mass ratio of chlorogenic acid and temozolomide is 100:100 or 200:100.

The present invention further provides the method for preparation of the pharmaceutical composition mentioned above, that is a commonly used pharmaceutical preparation obtained by using chlorogenic acid and temozolomide as active ingredients, with addition of pharmaceutically acceptable excipients.

Wherein, the preparation is an oral preparation or an injection preparation.

The present invention further provides the use of the the pharmaceutical composition mentioned above in the preparation of drugs for treatment of high-grade brain tumors that recur after surgery and do not response to standard treatment.

The present invention further provides the use of chlorogenic acid in the preparation of drugs for treatment of high-grade brain tumors that recur after surgery and do not response to standard treatment.

Wherein, the brain tumors include glioblastoma, anaplastic astrocytoma, anaplastic oligodendrocytoma, and anaplastic oligodendroblastoma.

The high-grade brain tumors that recur after surgery and are ineffective after standard treatment in the present invention means those relapsing after surgery and are ineffective using standard therapy.

Standard treatment denotes surgery, chemotherapy, and radiotherapy in accordance with international and domestic guidelines (such as the Chinese Central Nervous System Glioma Diagnosis and Treatment Guidelines, Chinese Medical Journal, Vol. 96, Issue 7, Feb. 23, 2016). High-grade brain tumors denote brain tumors of grades III and IV in the WHO classification of central nervous system tumors.

The present invention provides a new method: treating patients with brain malignant tumors and recurrence of high-grade malignant brain tumors, as well as ineffective after international and domestic standard treatment (recurrence after surgery, radiotherapy, and chemotherapy, re-treatment as international and domestic guidelines being ineffective, disease progression), namely a) using injectable chlorogenic acid, the drug combination (temozolomide) and the pharmaceutical composition of oral chlorogenic acid for treatment of glioma; b. Injectable chlorogenic acid and oral chlorogenic acid are separately administrated.

The pharmaceutical composition or combination of the present invention; chlorogenic acid and temozolomide can be used at the same time or successively; it can treat patients who relapse after international and domestic standard treatment, are ineffective for further therapy, and have progressed disease. The pharmaceutical composition can effectively control, reduce, and eliminate the focus of malignant brain tumors, and significantly prolong the survival period of patients with malignant brain tumors (especially high-grades).

Obviously, based on above content of the present invention, according to the common technical knowledge and the conventional means in the field, without department from above basic technical spirits, other various modifications, alternations or changes can further be made.

By following specific examples of said embodiments, above content of the present invention is further illustrated. But it should not be construed that the scope of above subject of the present invention is limited to following examples. The techniques realized based on above content of the present invention are all within the scope of the present invention.

### EXAMPLES

The starting materials and equipment used in the specific examples of the present invention are all known products and can be obtained by purchasing commercially available products.

### Example 1

### The Drug Combination of the Present Invention

1 Chlorogenic Acid Injection

350 g chlorogenic acid was aseptically weighed, to which was added 850 g mannitol, and then subpacked as injections after preparation.

2 Temozolomide Capsules

100 g temozolomide was aseptically weighed and subpacked as capsules.

3 Instructions

Temozolomide was first orally administrated, and then chlorogenic acid injection was successively given according to the mass ratio of 300 (chlorogenic acid):150 (temozolomide).

### Example 2

### The Drug Combination of the Present Invention

1 Oral Tablets of Chlorogenic Acid

350 g chlorogenic acid was aseptically weighed, to which were added 850 g mannitol and 250 g microcrystalline cellulose, followed by pressing to obtain oral tablets.

2 Temozolomide Capsules

100 g temozolomide was aseptically weighed and subpacked as capsules.

3 Instructions

Temozolomide was first orally administrated, and then chlorogenic acid tablets was successively given according to the mass ratio of 300 (chlorogenic acid):150 (temozolomide).

### Example 3

### The Drug Combination of the Present Invention

1 Chlorogenic Acid Injection

100 g chlorogenic acid was aseptically weighed, to which was added 650 g mannitol, and then subpacked as injections after preparation.

2 Temozolomide Capsules

100 g temozolomide was aseptically weighed and subpacked as capsules.

3 Instructions

Both temozolomide capsules and chlorogenic acid injections were simultaneously used according to the mass ratio of 100 (chlorogenic acid):100 (temozolomide).

4 Animal Model: Temozolomide-Resistant Animal Model

### Example 4

### The Drug Combination of the Present Invention

1 Chlorogenic Acid Injection

100 g chlorogenic acid was aseptically weighed, to which were added 250 g mannitol and 6 g sodium bisulfite, sterilized and filtered after preparation, and then subpacked and lyophilized as powder injection.

2 Temozolomide Capsules

100 g temozolomide was aseptically weighed and subpacked as capsules.

3 Instructions

Chlorogenic acid injection was first administrated, and then temozolomide capsules were successively given according to the mass ratio of 100 (chlorogenic acid):100 (temozolomide).

### Example 5

### The Drug Combination of the Present Invention

1 Chlorogenic Acid Injection

35 g chlorogenic acid was aseptically weighed, to which was added 100 g mannitol, and then dissolved in water for injection, filtered, sterilized, freeze-dried to obtain freeze-dried powder injection.

2 Temozolomide Capsules

100 g temozolomide was aseptically weighed and subpacked as capsules.

3 Instructions

Chlorogenic acid powder injection was first administrated, and then temozolomide capsules were successively given according to the mass ratio of 35 (chlorogenic acid):100 (temozolomide).

### Example 6

### The Formula for Oral Preparation of the Pharmaceutical Composition According to the Present Invention

1. Formula 1

Chlorogenic acid 35 g, temozolomide 100 g.

Preparative method: chlorogenic acid and temozolomide were aseptically weighed according to the formula, mixed thorougly, and aseptically subpacked as powders.

2. Formula 2

Chlorogenic acid 35 g, temozolomide 100 g, bulking agent 500 g, binding agent 5 g. Preparative method: chlorogenic acid, temozolomide, bulking agent, and binding agent were weighed according to the formula, granulated, sieved, and subpacked as granules.

3. Formula 3

Chlorogenic acid 100 g, temozolomide 100 g, bulking agent 500 g, binding agent 5 g, and lubricant 3 g.

Preparative method: chlorogenic acid, temozolomide, bulking agent, and binding agent were weighed according to the formula, granulated, sieved, and then lubricant was added, followed by pressing, to obtain tablets.

Above bulking agents were one or more of mannitol, lactose, starch, microcrystalline cellulose, and dextrin; the binding agents were sodium carboxymethylcellulose and PVP; the lubricants were magnesium stearate, talcum powder, and micro silica gel.

### Example 7

### The Formula for Injection of the Pharmaceutical Composition According to the Present Invention

1. Formula 1

Chlorogenic acid 35 g, temozolomide 100 g.

Preparative method: chlorogenic acid and temozolomide were aseptically weighed according to the formula, mixed thorougly, and aseptically subpacked as powder injection.

2. Formula 2

Chlorogenic acid 350 g, temozolomide 100 g.

Preparative method: chlorogenic acid and temozolomide were weighed according to the formula, dissolved in water for injection, filtered, sterilized, and freeze-dried to obtain freeze-dried powder injection.

3. Formula 3

Chlorogenic acid 100 g, temozolomide 100 g, support agent 2667 g, and antioxidant 67 g.

Preparative method: chlorogenic acid, temozolomide, support agent, and antioxidant were weighed according to the formula, dissolved in water for injection, filtered, sterilized, and freeze-dried to obtain freeze-dried powder injection.

Said support agents were mannitol, lactose and glucose; the antioxidants were sodium bisulfate, vitamin, glutathione, and folic acid.

In the following, the beneficial effect of the present invention was proved by experimental examples:

### Experimental Example 1

### Clinical Statistics

This experiment investigated the efficacy of chlorogenic acid for injection in patients with high-grade (WHO III-8981 IV grades) glioma relapsed after failure of standard treatment.

1. Necessary Conditions of Enrolling Subjects

A total of 23 subjects completed the test in this trial, including 5 patients with grade III gliomas and 18 patients with grade IV gliomas. Among 23 cases, 11 cases had KPS≥60 points, and 12 cases KPS <60 points.

The subjects included in this trial were all patients with high-grade glioma who relapsed after surgery and didn't response to the standard therapy. The standard treatment was chemotherapy and radiotherapy. Among them, for chemotherapy temozolomide was administrated at an oral dose of 50-50 mg/m², and the administration cycle was 4-30, wherein 58% of subjects had 10 treatment cycles; while for radiotherapy 60-200GY was used, and chemotherapy was carried out for 10-50 times. Ineffective means tumor progression.

Administration mode and dosage: chlorogenic acid for injection was administered by intramuscular injection, and each chlorogenic acid (30 mg) for injection was dissolved in 0.5 ml saline (when normal saline cannot be used, 5% glucose injection can be used as a replacement), then intramuscularly injected at a dose of 2.0 mg/kg-7.0 mg/kg for 28 consecutive days, as a dosing cycle, wherein the dosing cycle was 1-10; after injection of chlorogenic acid, according to the patient's conditions, temozolomide was orally administered at 50-150 mg/m²/d for 7 consecutive days, and the drug was withdrawn for 7 days, wherein 2 weeks was a dosing cycle, and the dosing cycle was 0-10.

2. Preliminary Effectiveness Evaluation of Chlorogenic Acid for Injection

2.1 Evaluation Criterion

The effectiveness evaluation was performed according to the RANO standard, and the specific RANO standards are as follows:

| | Complete | | | |
|---|---|---|---|---|
| Items | remission | Part remission | Stability | Progession |
| T1 + enhance | Not found | Reducing ≥50% | Change from −50% | Increasing ≥25% |
| | | | to +25% | |
| T2/FLAIR | Stabilizing or | Stabilizing or | Stabilizing or | Stabilizing or |
| | decreasing | decreasing | decreasing | decreasing |
| New lesions | Not found | Not found | Not found | Not found |
| Corticosteroid | No need | Stabilizing or | Stabilizing or | Not as a |
| application | | decreasing | decreasing | standard |
| Clinical features | Stabilizing or | Stabilizing or | Stabilizing or | progession |
| | improving | improving | improving | |
| Conditions | All of the | All of the | All of the | Any of above |
| required for | above | above | above | items |
| evaluation | | | | |
| criterion | | | | |

Evaluation Criterions:

(1) Complete Remission (CR)

All the following conditions should be met: all measurable and unmeasurable enhanced lesions completely disappeared for more than 4 weeks; no new lesions; non enhanced lesions (T2 or FLAIR images) were stable or improved; patients were allowed to stop using hormone or only use physiological replacement; clinical symptoms were stable or improved.

(2) Part Remission (PR)

All the following conditions should be met: compared with the baseline before treatment, the sum of the products of the two vertical diameters of all measurable lesions decreased by ≥50%, and lasted for more than 4 weeks; non-measurable lesions did not progress; no new lesions; non-enhanced lesions (T2 or FLAIR images) were stable or improved under the same dose or lower dose of hormone; and the clinical symptoms of patients were stable or improved.

(3) Stable Disease (SD):

If the patient did not meet complete remission, partial remission or disease progression, and met all of the following conditions, the disease was considered stable: compared with the baseline before treatment, under the same dose or lower dose of hormones, non-enhancing lesions (T2 image or FLAIR image) were stable, and clinical symptoms were stable.

If the patient needed to increase the hormone dose due to worsening clinical symptoms or signs without disease progression confirmed by imaging, but the disease progression was confirmed in the subsequent MRI review, the time point when the disease was stable should be the last time when the disease was confirmed to be stable by imaging scan without increasing the hormone dose.

(4) Disease Progression (PD):

Meeting any one of the followings: under the conditions of the same or increasing hormone dosage, compared with the baseline before treatment (if the lesion was not reduced) or the smallest enhanced lesion when the response was optimal, the sum of the product of the two vertical diameters of the enhanced lesion increased >25%; non-enhanced lesions on T2 image or FLAIR image are significantly enlarged, but other complications (radiotherapy, demyelination, infection, postoperative changes, ischemic injury, epilepsy, etc.) factors should be excluded; any new lesions; non-measurable lesions had obvious progress; clinical symptoms had significantly deteriorated, but non-tumor factors (epilepsy, adverse drug reactions, complications of treatment, cerebrovascular events, infections, etc.) or hormone dose change factors should be excluded; due to clinical death or deterioration, the follow-up assessment cannot be completed.

2.2 Evaluation Results

Till database lock-up time (Feb. 5, 2018), preliminary effectiveness evaluation of chlorogenic acid for injection was carried out. A total of 26 subjects were enrolled in the trial, 3 cases were released early, and 23 cases can be counted:

(1) Effectiveness Evaluation of RANO Standard Enrollment

For all subjects: During phase I clinical trial, after one administration cycle, 12 cases of SD (among 12 cases, 5 patients having a lesion reduction); 11 cases of PD; during phase I clinical trial, after one cycle of administration, DCR (Disease control rate) was 52.17%; after two cycles of administration, DCR was 33.33%.

After six cycles of administration, No. 001 subject was evaluated as CR and No. 010 subject was evaluated as PR, and the result was encouraging. Most patients with relapsed high-grade glioma, on whom standard treatments failed, will in the irreversible state of SD-PD-death, and it was rare for CR or PR to occur (small probability events). Therefore, in this trial, two of the 23 subjects experienced CR and PR, and the significant therapeutic response far exceeded the expected of researchers.

For 18 patients with grade IV brain tumors, the correlation of the administration cycle and the survival were analyzed. 18 patients with grade IV glioma who completed the trial were divided into survival and death for statistics. Preliminary results showed that nine dead subjects did not respond significantly to chlorogenic acid, with a response rate of 11.1% (9 dead subjects with class IV glioblastoma took drug for one cycle, and only one patient had a tumor assessment result of SD, while eight patients had PD). 9 survival subjects with grade IV glioblastoma had a higher response to chlorogenic acid, with a response rate of 88.9% (Among 9 survival subjects with grade IV glioblastoma, after administration of the drug for 1 cycle, eight subjects had a tumor assessment result of SD, while one subject had PD), still undergoing survival follow-up.

A total of 9 subjects were enrolled in the 3.0 mg/kg dose group. After one cycle of administration, the tumors in three subjects shrank (but not reaching PR). Among three subjects with tumor shrinkage, after two cycles of administration, the target intracranial lesion in one subject disappeared, but due to the progression of the lung lesion, the comprehensive assessment was SD, and DCR after 1 cycle of administration was 66.67%.

(2) Median OS

As the obtained trial data, the median OS (starting from randomization) for the 3.0 mg/kg dose group was 9.3 months; the median OS (starting after recurrence) was 13.2 months. According to the standards reported in the literature, the median OS after recurrence based on prior treatment methods was 7.2-9.6 months, while the drug of the present invention had exceeded the approved standard of 3.6 months reported in the literature.

Nowadays (2018 Feb. 5), for nine subjects in the 3.0 mg/kg group, only one subject died, and one subject was lost to follow-up. The median OS of 50% deaths had not yet been reached, and the current survival subjects were still in continuous follow-up, thus the final median OS of 3.0 mg/kg dose group should be much higher than the currently reported value.

Experimental results proved that chlorogenic acid or the combination of chlorogenic acid and temozolomide could effectively treat high-grade brain tumors that relapsed after surgery and on which standard treatments had failed, and prolonged the survival period of patients with high-grade brain tumors that relapsed after standard treatments.

### Experimental Example 2

### Animal Experiment

1. Cell Culture for High-Grade Brain Tumor that Recurred After Surgery and on which the Standard Therapy Failed

1.1 Cell culture: The pathological tissue for cell culture was a tissue sample removed surgically from high-grade brain tumor that relapsed after surgery and on which the standard therapy failed. Glioblasts cells, high-grade anaplastic astrocytoma cells, high-grade anaplastic oligodendrocytoma cells, high-grade anaplastic oligoastrocytoma cells, and brain tumor cells with unclear pathological types, obtained by separation and culture of pathological tissue, were seeded in DMEM complete culture medium containing 10% FBS and routinely incubated in a 37° C., 5% carbon dioxide incubator under saturated humidity. TMZ was dissolved in DMSO solution, and the mass concentration of the stock solution was 100 mg/mL. Tumor cells in the logarithmic growth phase were diluted to obtain a cell suspension at 1×10⁵ cells/mL, and the initial inducer dose 0.25 μg/mL of TMZ was added after routinely culturing for 24 h. The culture was continued for 15-20 days, and after stabilization of cell growth, the induced concentration of the drug was doubled, then the cells were further cultured for 15-20 d at each induced concentration. Thus, the final concentration of TMZ was 16.00 μg/mL, and various pathological types of cell suspensions were obtained.

2. Tumor Inhibition Rate of the Combination of Chlorogenic Acid and Temozolomide in Animal Model of High-Grade Brain Tumor that Recured After Surgery and on which the Standard Therapy Failed

2.1 Tumor Inhibition Rate of the Combination of Chlorogenic Acid and Temozolomide in Animal Model of Glioblastoma that Relapsed After Surgery and on which the Standard Therapy Failed

The cell suspension obtained in step 1 was inoculated into rats in situ, to prepare animal models of glioblastoma that relapsed after surgery and on which the standard therapy failed, and the animals were divided into test groups listed in the following table. Chlorogenic acid injections were administered intraperitoneally at 20 mg/kg or 35 mg/kg daily for 10 days, and temozolomide was orally administered at 10 mg/kg or 20 mg/kg for 5 days. On the 5^{th} day after drug-withdrawal, the size of the in situ tumor in the rat brain was observed, and the tumor was weighed. The tumor inhibition rate was calculated.

| | | | | Tumor |
|---|---|---|---|---|
| | Dose | Number of | Tumor | inhibition |
| Groups | (mg/kg) | animals (n) | weight | rate (%) |
| Chlorogenic | 7 + 20 | 10 | 0.65 ± 0.07 | 68.2 |
| acid + temozolomide | | | | |
| Chlorogenic | 20 + 20 | 10 | 0.32 ± 0.05 | 84.3 |
| acid + temozolomide | | | | |
| Chlorogenic | 20 + 10 | 10 | 0.72 ± 0.08 | 64.0 |
| acid + temozolomide | | | | |
| Chlorogenic | 35 + 10 | 10 | 0.55 ± 0.06 | 73.1 |
| acid + temozolomide | | | | |
| Chlorogenic acid | 35 | 10 | 0.98 ± 0.09 | 52.2 |
| temozolomide | 20 | 9 | 1.54 ± 0.10 | 24.1 |
| temozolomide | 10 | 8 | 1.75 ± 0.14 | 14.6 |
| Blank control | — | | 2.05 ± 0.25 | |

As shown in above table, chlorogenic acid alone had a significant inhibitory effect on glioblastomas that relapsed after surgery and on which the standard therapy failed, but when temozolomide was used alone, the effect was poor; the use of chlorogenic acid combined with temozolomide could further improve the therapeutic effect. Amongst, the effect was particularly good when the doses of chlorogenic acid and temozolomide were both 20 mg/kg. In this group, the weight ratio of chlorogenic acid and temozolomide was 2:1.

3.2 Tumor Inhibition Rate of the Combination of Chlorogenic Acid and Temozolomide in Animal Model of High-Grade Anaplastic Astrocytoma that Relapsed After Surgery and on which the Standard Therapy Failed

The cell suspension obtained in step 1 was inoculated into rats in situ, to prepare animal models of high-grade anaplastic astrocytoma that relapsed after surgery and on which the standard therapy failed, and the animals were divided into test groups listed in the following table. Chlorogenic acid injections were administered intraperitoneally at 20 mg/kg or 35 mg/kg daily for 10 days, and temozolomide was orally administered at 10 mg/kg or 20 mg/kg for 5 days. On the 5^{th} day after drug-withdrawal, the size of the in situ tumor in the rat brain was observed, and the tumor was weighed. The tumor inhibition rate was calculated.

| | | | | Tumor |
|---|---|---|---|---|
| | Dose | Number of | Tumor | inhibition |
| Groups | (mg/kg) | animals (n) | weight | rate (%) |
| Chlorogenic | 7 + 20 | 10 | 0.85 ± 0.08 | 62.2 |
| acid + temozolomide | | | | |
| Chlorogenic | 20 + 20 | 10 | 0.45 ± 0.04 | 80.0 |
| acid + temozolomide | | | | |
| Chlorogenic | 20 + 10 | 10 | 0.93 ± 0.08 | 58.7 |
| acid + temozolomide | | | | |
| Chlorogenic | 35 + 10 | 10 | 0.73 ± 0.07 | 67.5 |
| acid + temozolomide | | | | |
| Chlorogenic acid | 35 | 10 | 1.12 ± 0.10 | 50.2 |
| temozolomide | 20 | 9 | 1.78 ± 0.11 | 20.9 |
| temozolomide | 10 | 9 | 1.95 ± 0.18 | 13.3 |
| Blank control | | | 2.25 ± 0.20 | |

As shown in above table, chlorogenic acid alone had a significant inhibitory effect on high-grade anaplastic astrocytoma that relapsed after surgery and on which the standard therapy failed, but when temozolomide was used alone, the effect was poor; the use of chlorogenic acid combined with temozolomide could further improve the therapeutic effect. Amongst, the effect was particularly good when the doses of chlorogenic acid and temozolomide were both 20 mg/kg. In this group, the weight ratio of chlorogenic acid and temozolomide was 2:1.

3.3 Tumor Inhibition Rate of the Combination of Chlorogenic Acid and Temozolomide in Animal Model of High-Grade Anaplastic Oligodendrocytoma that Relapsed After Surgery and on which the Standard Therapy Failed

The cell suspension obtained in step 1 was inoculated into rats in situ, to prepare animal models of high-grade anaplastic oligodendrocytoma that relapsed after surgery and on which the standard therapy failed, and the animals were divided into test groups listed in the following table. Chlorogenic acid injections were administered intraperitoneally at 20 mg/kg or 35 mg/kg daily for 10 days, and temozolomide was orally administered at 10 mg/kg or 20 mg/kg for 5 days. On the 5^{th} day after drug-withdrawal, the size of the in situ tumor in the rat brain was observed, and the tumor was weighed. The tumor inhibition rate was calculated.

| | | | | Tumor |
|---|---|---|---|---|
| | Dose | Number of | Tumor | inhibition |
| Groups | (mg/kg) | animals (n) | weight | rate (%) |
| Chlorogenic | 7 + 20 | 10 | 0.72 ± 0.06 | 66.2 |
| acid + temozolomide | | | | |
| Chlorogenic | 20 + 20 | 10 | 0.36 ± 0.04 | 83.1 |
| acid + temozolomide | | | | |
| Chlorogenic | 20 + 10 | 10 | 0.69 ± 0.07 | 67.6 |
| acid + temozolomide | | | | |
| Chlorogenic | 35 + 10 | 10 | 0.58 ± 0.06 | 72.8 |
| acid + temozolomide | | | | |
| Chlorogenic acid | 35 | 10 | 1.01 ± 0.08 | 52.6 |
| temozolomide | 20 | 9 | 1.65 ± 0.14 | 22.5 |
| temozolomide | 10 | 8 | 1.80 ± 0.15 | 15.5 |
| Blank control | | | 2.13 ± 0.22 | |

As shown in above table, chlorogenic acid alone had a significant inhibitory effect on high-grade anaplastic oligodendrocytoma that relapsed after surgery and on which the standard therapy failed, but when temozolomide was used alone, the effect was poor; the use of chlorogenic acid combined with temozolomide could further improve the therapeutic effect. Amongst, the effect was particularly good when the doses of chlorogenic acid and temozolomide were both 20 mg/kg. In this group, the weight ratio of chlorogenic acid and temozolomide was 2:1.

3.4 Tumor Inhibition Rate of the Combination of Chlorogenic Acid and Temozolomide in Animal Model of High-Grade Anaplastic Oligoastrocytoma that Relapsed After Surgery and on which the Standard Therapy Failed

The cell suspension obtained in step 1 was inoculated into rats in situ, to prepare animal models of high-grade anaplastic oligoastrocytoma that relapsed after surgery and on which the standard therapy failed, and the animals were divided into test groups listed in the following table. Chlorogenic acid injections were administered intraperitoneally at 20 mg/kg or 35 mg/kg daily for 10 days, and temozolomide was orally administered at 10 mg/kg or 20 mg/kg for 5 days. On the 5^{th} day after drug-withdrawal, the size of the in situ tumor in the rat brain was observed, and the tumor was weighed. The tumor inhibition rate was calculated.

| | | | | Tumor |
|---|---|---|---|---|
| | Dose | Number of | Tumor | inhibition |
| Groups | (mg/kg) | animals (n) | weight | rate (%) |
| Chlorogenic | 7 + 20 | 10 | 0.62 ± 0.05 | 68.7 |
| acid + temozolomide | | | | |
| Chlorogenic | 20 + 20 | 10 | 0.30 ± 0.04 | 84.8 |
| acid + temozolomide | | | | |
| Chlorogenic | 20 + 10 | 10 | 0.70 ± 0.06 | 64.6 |
| acid + temozolomide | | | | |
| Chlorogenic | 35 + 10 | 10 | 0.51 ± 0.05 | 74.2 |
| acid + temozolomide | | | | |
| Chlorogenic acid | 35 | 10 | 1.05 ± 0.07 | 47.0 |
| temozolomide | 20 | 8 | 1.58 ± 0.12 | 20.2 |
| temozolomide | 10 | 8 | 1.72 ± 0.14 | 13.1 |
| Blank control | | | 1.98 ± 0.16 | |

As shown in above table, chlorogenic acid alone had a significant inhibitory effect on high-grade anaplastic oligoastrocytoma that relapsed after surgery and on which the standard therapy failed, but when temozolomide was used alone, the effect was poor; the use of chlorogenic acid combined with temozolomide could further improve the therapeutic effect. Amongst, the effect was particularly good when the doses of chlorogenic acid and temozolomide were both 20 mg/kg. In this group, the weight ratio of chlorogenic acid and temozolomide was 2:1.

3.5 Tumor Inhibition Rate of the Combination of Chlorogenic Acid and Temozolomide in Animal Model of Brain Tumors with Unclear Pathological Types that Relapsed After Surgery and on which the Standard Therapy Failed

The cell suspension obtained in step 1 was inoculated into rats in situ, to prepare animal models of brain tumor with unclear pathological types that relapsed after surgery and on which the standard therapy failed, and the animals were divided into test groups listed in the following table. Chlorogenic acid injections were administered intraperitoneally at 20 mg/kg or 35 mg/kg daily for 10 days, and temozolomide was orally administered at 10 mg/kg or 20 mg/kg for 5 days. On the 5^{th} day after drug-withdrawal, the size of the in situ tumor in the rat brain was observed, and the tumor was weighed. The tumor inhibition rate was calculated.

| | | | | Tumor |
|---|---|---|---|---|
| | Dose | Number of | Tumor | inhibition |
| Groups | (mg/kg) | animals (n) | weight | rate (%) |
| Chlorogenic | 7 + 20 | 10 | 0.69 ± 0.05 | 68.3 |
| acid + temozolomide | | | | |
| Chlorogenic | 20 + 20 | 10 | 0.36 ± 0.05 | 83.5 |
| acid + temozolomide | | | | |
| Chlorogenic | 20 + 10 | 10 | 0.75 ± 0.06 | 65.6 |
| acid + temozolomide | | | | |
| Chlorogenic | 35 + 10 | 10 | 0.57 ± 0.07 | 73.9 |
| acid + temozolomide | | | | |
| Chlorogenic acid | 35 | 10 | 1.21 ± 0.11 | 44.5 |
| temozolomide | 20 | 9 | 1.62 ± 0.14 | 25.7 |
| temozolomide | 10 | 9 | 1.83 ± 0.13 | 16.0 |
| Blank control | | | 2.18 ± 0.23 | |

As shown in above table, chlorogenic acid alone had a significant inhibitory effect on brain tumors with unclear pathological types that relapsed after surgery and on which the standard therapy failed, but when temozolomide was used alone, the effect was poor; the use of chlorogenic acid combined with temozolomide could further improve the therapeutic effect. Amongst, the effect was particularly good when the doses of chlorogenic acid and temozolomide were both 20 mg/kg. In this group, the weight ratio of chlorogenic acid and temozolomide was 2:1.

The experiment proved that chlorogenic acid had good therapeutic effects on high-grade brain tumors, including high-grade anaplastic astrocytoma, high-grade anaplastic oligodendrocytoma, high-grade anaplastic oligoastrocytoma and the same, and the effect was better than temozolomide. However, the combined use of chlorogenic acid and temozolomide could further improve the therapeutic effect.

In summary, for the pharmaceutical composition or the combined drug of the present invention, chlorogenic acid and temozolomide could have a synergistic effect and show excellent curative effect; treat high-grade brain tumors that relapsed after surgery and on which the standard therapy failed, prolong the survival period of patients, and have good clinical application prospects.

1. A drug combination for treatment of high-grade brain tumors that recur after surgery and do not response to standard treatment, characterized in that said combination contains unit preparations of the same or different specifications, used for simultaneous or separate administration of chlorogenic acid and temozolomide, as well as pharmaceutically acceptable carrier.

2. The drug combination according to claim 1, characterized in that the mass ratio of chlorogenic acid and temozolomide is 35:100-350:100.

3. The drug combination according to claim 2, characterized in that the mass ratio of chlorogenic acid and temozolomide is 85:100-200:100; the mass ratio of chlorogenic acid and temozolomide is 85:100-175:100; preferably, the mass ratio of chlorogenic acid and temozolomide is 100:100 or 200:100.

4. The drug combination according to claim 1, characterized in that the preparation is an oral preparation or an injection preparation.

5. The use of the drug combination according to claim 1 in the preparation of drugs for treatment of high-grade brain tumors that recur after surgery and do not response to standard treatment.

6. The use according to claim 5, characterized in that the brain tumors include glioblastoma, anaplastic astrocytoma, anaplastic oligodendrocytoma, and anaplastic oligodendroblastoma.

7. A pharmaceutical composition, characterized in that it contains chlorogenic acid and temozolomide.

8. The pharmaceutical composition according to claim 7, characterized in that the mass ratio of chlorogenic acid and temozolomide is 35:100-350:100.

9. The pharmaceutical composition according to claim 8, characterized in that the mass ratio of chlorogenic acid and temozolomide is 85:100-200:100; the mass ratio of chlorogenic acid and temozolomide is 85:100-175:100; preferably, the mass ratio of chlorogenic acid and temozolomide is 100:100 or 200:100.

10. The method for preparation of the pharmaceutical composition according to claim 7, characterized in that it is a commonly used pharmaceutical preparation obtained by using chlorogenic acid and temozolomide as active ingredients, with addition of pharmaceutically acceptable excipients.

11. The method according to claim 10, characterized in that the preparation is an oral preparation or an injection preparation.

12. The use of the pharmaceutical composition according to claim 7 in the preparation of drugs for treatment of high-grade brain tumors that recur after surgery and do not response to standard treatment.

13. The use of chlorogenic acid in the preparation of drugs for treatment of high-grade brain tumors that recur after surgery and do not response to standard treatment.

14. The use according to claim 12, characterized in that the brain tumors include glioblastoma, anaplastic astrocytoma, anaplastic oligodendrocytoma, and anaplastic oligodendroblastoma.
